(19) United States
(12) Patent Application Publication
(10) Pub. No.: US 2005/0154470 A1
(43) Pub. Date: Jul. 14, 2005

(54) Modular phrosthesis assembly including tapered adjustments

(76) Inventor: Ronald Sekel, Matraville (AU)

Correspondence Address: STITES & HARBISON PLLC, 1199 NORTH FAIRFAX STREET, SUITE 900, ALEXANDRIA, VA, 22314, US

(21) Appl. No.: 10/503,731

(22) PCT Filed: Feb. 7, 2003
(86) PCT No.: PCT/AU03/00122

(30) Foreign Application Priority Data
Feb. 8, 2002 (AU) PS 0388

(51) Int. Cl. A61F 2/38

(52) U.S. Cl. 623/20.15; 623/20.28

(57) ABSTRACT

A prosthesis assembly for implantation in a skeletal site; the assembly comprising: a first component (8) for fixation in a bone cavity (9), a second component (7) capable of direct or indirect engagement with the first component; at least one adaptor which engages the first and second components thereby allowing adjustment of the second component from a first disposition of the second component relative to a predetermined reference.

### BACKGROUND

The present invention relates to improvements in surgical prostheses and more particularly relates to a prosthesis assembly including mutually interacting tapered adaptor elements capable of use in knee and other skeletal prostheses and which allow during implantation, fine adjustability of at least one component of the prosthesis assembly through at least five degrees of freedom namely; rotation about X, Y and Z axes, vertical adjustment along the Z axis. Although the assembly will primarily be described with reference to its application in adjustment of knee prostheses and particularly tibial and femoral components, it will be appreciated by persons skilled in the art that the double taper arrangements to be described may be applied in other prostheses at skeletal sites such as but not limited to shoulders, hips, ankles, fingers and in dental applications.

### PRIOR ART

Knee arthroplasty is a well-known surgical procedure by which a diseased and/or damaged natural knee joint is replaced by a prosthetic knee joint. Typical knee prostheses include a tibial component, a femoral component and a patellar component. Modern total knee replacement involves the resurfacing of the femoral condyles with a metallic component, roughly approximating the shape of the anatomical femoral condyles, and resurfacing the tibial plateau with usually, but not exclusively, a polyethylene component having a metallic tibial base plate. Optimal conformity between the polyethylene of the tibial component and the metallic femoral component has in the past been a problem area. Ideally the femoral component should be congruent with the top of the tibial component in order to minimise wear of a surface liner which is usually polyethylene. The difficulty, however, is that the knee joint does not act as a fixed axis hinge. During normal movements of the knee, rotation of the femur upon the tibia occurs, and roll back of the femoral condyles upon the tibia occurs, particularly when the knee is flexed. The provision of a bearing in the form of a cam mechanism between the femoral component and the polyethylene tibial component means that with increased flexion of the knee increased posterior translation of the femoral component upon the tibia occurs, the bearing between the tibial and femoral components is incongruent and therefore theoretically undesirable, resulting in high contact stress, leading to increased wear of the surface liner which is usually plastics. For example, if the plane of the tibial plate when fitted to the tibia is misaligned with the resected proximal surface of tie tibia, uneven wear will result between the articular surfaces. A patient may not notice the misalignment and uneven loading of the femoral component on the tibial component but where the loading is concentrated through one condyle wear is accelerated. This may lead to a reduction of up to 50% of the normal life of the prosthesis.

The femoral component generally includes a pair of spaced apart condylar portions, the superior surfaces of which articulate with a portion of the polyethylene tibial component. A femoral stem assembly, used to provide lateral stability to the replaced knee joint, seats within the medullary cavity of a distal portion of a femur, and is typically fixed to the femoral component by specialized fixation, such as a collar and bolt. Some prosthetic knee joints include a taper which may be a Morse taper, that extends from the back surface of the femoral component to mate with a femoral sleeve that is securable to the femoral stem assembly.

A femoral sleeve, which helps to fill spaces at the opening of the medullary canal, can also provide for a modular assembly allowing a surgeon to select the most appropriate femoral stem from a selection of stems having different lengths and diameters for attachment to one of a selection of femoral components. This modular configuration significantly reduces the number of individual components that must be purchased, stocked, and used during a surgical procedure. Although the femoral stem, whatever its dimensions, is usually angled with respect to the inferior surface of the femoral component and either off-set anteriorially/posterially or at a central location, it is sometimes desirable to orient the femoral stem perpendicularly with respect to the back surface. For example, depending on particular patient requirements, the femoral stem may need to be offset fore or aft with respect to the front of the femoral component. Similarly, the femoral stem may need to be angled varying degrees to the left or right with respect to the front plane of the femoral component. A Morse type taper post, is integrally cast as part of the femoral component. Furthermore, there is a requirement for a range of sizes of the overall femoral component. Therefore, in order to accommodate all of the possible combinations of overall femoral component size, fore/neutral/aft positioning of the Morse type taper post, and left/perpendicular/right angling of the Morse type taper post, a doctor or hospital is required to maintain an undesirably substantial stock of knee prosthesis components. Despite the existence of knee joint prostheses having modular components, there remains a need for a modular knee joint prosthesis assembly that has greater versatility of adjustment to accommodate differing patient anatomy and a maligned components.

An example of a known knee prosthesis arrangement is disclosed in U.S. Pat. No. 5,593,449 to Robertson Jr. That patent discloses a dual taper stem extension for knee prosthesis for surgical implantation to a patient's leg bone at the knee joint area. The prosthesis includes a prothesis body portion that extends transversely relative to the patients intramedullary canal for carrying a bearing surface that articulates with the patient's adjacent leg bone or with another prosthesis component. A conical connector extends from the prosthesis portion and along an ads that generally tracks the patient's intramedullary canal. A stem member includes first and second end portions and has a central longitudinal stem axis. The stem member includes a socket at each end portion for forming connections to the conical connector at the respective end portions as selected by the surgeon. One of the sockets has a central longitudinal axis that generally coincides with the central longitudinal axis of the stem. The other socket has a central longitudinal axis that forms an acute angle with the axis of the stem. The arrangement disclosed in this patent allows the surgeon to select from a choice of two taper angles the valgus angle for a stem extension that will best fit the patients intramedullary canal but once the angle is selected the coupling allows only two degrees of freedom i.e. axial and rotational movement.

Another known knee prosthesis is disclosed in U.S. Pat. No. 5,782,921 to Colleran which teaches a modular knee prosthesis including a Morse taper post that is matable with a first portion of a femoral sleeve. A second portion of the femoral sleeve is joined with a femoral stem that is introducible within the medullary canal of a distal portion of a femur. The modular knee prosthesis includes a femoral component, a bolt, and a Morse taper post. The femoral component has a superior surface, an inferior surface, and an aperture. The bolt includes a head portion engagable with the superior surface of the femoral component to inhibit movement of the bolt through the femoral component, and an elongate shaft portion that extends from the head portion of the bolt. The elongate shaft portion has a length sufficient to protrude through the aperture beyond the inferior surface of the femoral component. The Morse taper post is engagable with the elongate shaft portion of the bolt to retain the Morse taper post in a fixed position with respect to the femoral component and the distal end of the Morse taper post is introducible within a femoral sleeve.

U.S. Pat. No. 5,800,552 teaches a Mechanically linked hinged total knee prosthesis. A resurfacing type of total knee prosthesis is disclosed which also provides a posterior stabilization function over the entire range of flexion. The knee prosthesis provides primary or supplementary posterior stabilization of the reconstructed knee joint by means of a unique mechanical cam/follower mechanism, which is integrated within the medial and lateral distal condyles of the femoral component to provide functional compensation for lost, resected or incompetent posterior cruciate ligaments or to work in conjunction with surgically retained viable or questionably viable cruciate ligament structures of the reconstructed knee joint. The invention extends to prostheses including a hinge connection that defines a posterior stabilization construction separate from that defined by the condyles. One embodiment of the invention extends individually to the posterior stabilizing hinge assembly.

Another knee prosthesis disclosed in U.S. Pat. No. to McMinn comprises a femoral component, a tibial component and a meniscal component therebetween, a stabilising peg extending from the tibial component through an elongated slot in the meniscal component and into an opening in the femoral component between a pair of condylar members thereof. The part of the peg extending through the slot allows the meniscal component to rotate and also to move linearly about the peg along one path, whilst the part of the peg in said opening engages cam surfaces on a projection between said condylar members as the knee is flexed, in use, and said linear movement of the meniscal component occurs.

Typically a knee prosthesis will comprise a femoral component for securing to the femur, an opening defined by the femoral component, a tibial component for securement to the tibia, an opening through the tibial component, a bearing component between the femoral and tibial components, the femoral component and the bearing component having respective curved articulatory bearing surfaces of congruent form, an elongated slot in the bearing component, a locator separate from the tibial component, a stem part of the locator extending from an enlarged part thereof, the stem part extending through the opening in the tibial component, through the elongated slot in the bearing component, and into the opening defined by the femoral component, the bearing component being capable of rotational movement about the locator, and the elongated slot in the bearing component having a width, such as to prevent relative lateral movement between the locator and the bearing component, and a length to allow linear movement of the bearing component relative to the locator along one path. The linear movement occurs, in use, upon flexion of the knee, and the enlarged part of the locator being disposed at an opposite side of the tibial component to that at which the bearing component engages, and being oversized relative to said opening through the tibial component so as to prevent passage of said enlarged part therethrough. Examples of resurfacing types of total knee prosthetic devices are also disclosed in the following U.S. patents incorporated by reference herein. U.S. Pat. No. 3,774,244 to Walker, U.S. Pat. No. 3,728,742 to Averill et al. U.S. Pat. No. 4,081,866 and U.S. Pat No. 4,207,627 to Cloutier.

Although the issue of and need for greater versatility of adjustment of prostheses has been addressed in a number of prior art arrangements such as those described above, there is still a need to increase the adjustability of artificial joints relative to orthogonal XY and Z axes and rotationally through multiple thee dimensional degrees of freedom to more easily compensate for unwanted misalignments.

### INVENTION

There is a long felt want in the art to provide a convenient means for fine adjustments of prostheses, where an initial fit is not in conformity with agent parameters. For example, in the case of a tibial component of a knee prosthesis the tibial plate may not align with a patient reference plane. The misalignment may be in one or more planes or in one or more axes. According to present arrangements once the tibial component has been inserted as best the surgeon can, unwanted misalignments are tolerated due to the significant problems in resetting. Accurate fixation of the tibial component to ensure proper alignment is a difficult surgical objective particularly due to the difficulty in accurately preparing the medullary cavity in the tibia. In other bone sites and joints of the skeletal frame it would be an advantage if a surgeon could make fine axial, rotational, lateral and anterior/posterior adjustments through multiple planes and axes as this would allow correction of any misalignments or non conformity with insertion parameters.

The present invention provides an assembly including an adaptor which allows a surgeon to make fine adjustments to a component which directly or indirectly anchored in bone. The assembly is capable of use with a variety of bone and skeletal joint prosthesis. For instance the assembly and associated adaptors may be applied in effecting fine adjustments to dental fixations, tibial and femoral implants (distal or proximal), ankles, fingers and a variety of other joints and bone sites.

It is therefore an object of the invention to provide a modular prosthesis assembly including an adaptor which allows increased versatility of adjustment to accommodate predetermined insertion parameters, patient anatomy, joint attitude and conditions while maintaining a relatively low component count. It is another object of the invention to provide a modular prosthesis assembly including components that are physiologically and geometrically compatible with different anatomical conditions. Still another object of the invention is to provide a modular prosthesis that is suitable for use in bone sites and joints such as but not limited to shoulders and ankles, right and left knees. It is a farther object of the invention to provide an adaptor for use with a bone prosthesis and which allows adjustment of a component attached thereto through at least five degrees of freedom namely rotation about X Y and Z orthogonal axes, axial extension along the Z axis and displacement relative to X and Y axes, thereby enabling a surgeon to make fine realignment adjustments to the component to more accurately match the component with patient geometry.

Although the invention will be primarily described with reference to its application to knee prostheses it will be recognised by persons skilled in the art that the adaptor and associated taper arrangements described herein which allow five degrees of freedom for fine adjustments to the attitude of a component may be applied in other prostheses such as may be used to repair fingers, thumbs, shoulders and ankles. The assembly may also be used in dental applications where a component is used to anchor an artificial tooth to a jaw bone. It will be appreciated by persons skilled in the art that tapers other than a Morse type taper may be used on the assembly and adaptors according to the invention.

Typically, a known modular knee prosthesis includes a femoral component, a bolt and a Morse type taper post. The femoral component bas a front surface, a back surface, and an aperture extending there between. The bolt includes a head portion engagable with the front surface of the femoral component to inhibit movement of the bolt through the femoral component, and an elongate shaft portion that extends from the head portion of the bolt. The known tibial component of a knee prosthesis comprises a tibial plate which receives a polyethylene liner which provides an articular surface co operating wit the femoral component. Integral with the Tibial plate is a stem adapted for insertion in a medullary cavity of tibial bone. The stem is friction fitted and may be cemented into a suitably reamed medullary cavity. However if the reamed cavity is inaccurately formed, the tibial plate (or corresponding femoral component) may sit at an angle relative to a bone section cut by the surgeon as a reference prior to insertion of the tibial component. Once the known tibial component is inserted, the preferred way a correcting alignment adjustment may be made is to remove the tibial component and try to re set it. This is an undesirable solution to misalignment as a refit will possibly result in a potentially weaker bone/component bond.

According to one embodiment of the invention, there is provided a modular prosthesis assembly for use in but not limited to such joints as knees and shoulders wherein the assembly includes an anchoring member insertable in bone, at least one adaptor and a component set according to a predetermined reference and which simulates anatomical geometry; wherein the adaptor engages the anchoring member and the component to allow adjustment of said component in the event the component is misaligned with a predetermined anatomical reference.

According to another embodiment, the prosthesis assembly comprises an anchoring member insertable in a bone cavity, a tibial component which is capable of mating with the anchoring member; an adaptor capable of co operating with said anchoring member and the tibial component to allow fine adjustment of the tibial component.

According to a preferred embodiment, the fine adjustments of the tibial component may be axial, rotational about X,Y and Z axes or offset relative to a longitudinal axis.

In one broad form the present invention comprises:
a prosthesis assembly for implantation in a skeletal site; the assembly comprising;
a first component for fixation in a bone cavity, a second component capable of direct or indirect engagement with the first component;
at least one adaptor which engages said first and second components thereby allowing adjustment of the second component from a first disposition of the second component relative to a predetermined reference.

Preferably, the first component provides an anchorage in said bone for the assembly and receives the at least one adaptor, wherein the at least one adaptor joins the first component to the second component.

Preferably, the joining adaptor includes a body having an external tapered region and a tapered inner recess, wherein the external tapered region releasably engages the first component and the inner tapered recess receives therein the second component.

The second component is preferably adjustable through at least four degrees of freedom relative to said reference; namely laterally, angularly, axially or rotationally relative to X, Y and Z axes. The external tapered region is preferably symmetric relative to a longitudinal axis of said adaptor.

According to one embodiment the inner taper of the adaptor is co axial with the external taper. According to another embodiment, a longitudinal axis of the inner taper is disposed at an angle to a longitudinal axis of said adaptor.

The inner taper may be offset relative to but parallel to a longitudinal axis of said adaptor or the inner taper may be offset from and at an angle relative to a longitudinal axis of the adaptor. The first component includes a tapered recess which engages said external taper of said adaptor and the second component preferably comprises a tibial plate connected to a tapered stem. In another embodiment the first component is a femoral implant.

In another broad form, the present invention comprises;
a modular prosthesis assembly comprising; an anchorage component insertable in bone and a coupling component which co operates with said anchorage component to assume a first predetermined orientation relative to said anchorage component; the assembly further comprising an adaptor insertable between said anchorage component and said coupling component to allow a secondary adjustment of said coupling component relative to said first predetermined orientation of said coupling component.

Preferably, the anchorage member and the coupling member are capable of engagement with each other via male/female or female/male tapers. Preferably, the adaptor is engagable with the anchorage member and the coupling member via male/female or female/male tapers.

In another broad form the present invention comprises:

An adaptor for use with a prosthesis assembly for implantation in a skeletal site, the adaptor including a body having an external tapered region and an inner tapered recess, and wherein said external tapered region engages a corresponding tapered recess of a first implantable component of said assembly and the inner tapered recess receives therein a second component of said assembly.

The adaptor which is preferably cylindrical, allows adjustment of said second component relative to a first engaged position of said second component. In one embodiment, the inner taper is co axial with the outer taper. In another embodiment, the inner taper is disposed at an angle to a longitudinal axis of said adaptor. The inner taper may be offset from but parallel to a longitudinal axis of said adaptor. Alternatively, the inner taper is offset from and at an angle relative to a longitudinal axis of the adaptor.

In another form the present invention comprises;
a knee prosthesis comprising a femoral component for attachment to a femur, an opening defined by the femoral component, a tibial component for attachment to a tibia, an opening through the tibial component, a bearing component between the femoral and tibial components, the femoral component and the bearing component having respective curved articulatory bearing surfaces; the knee prosthesis further comprising; an adaptor capable of use with said tibial or femoral component wherein said adaptor enables secondary orthogonal, rotational lateral and axial adjustment of said tibial and femoral components.

In another broad form the present invention comprises:
a prosthesis assembly for implantation in a skeletal site; the assembly comprising;
a first anchorage component for fixation in a bone cavity, a second component capable of direct or indirect engagement with the first component;
wherein, the anchorage component comprises a tapered recess which receives a corresponding tapered member of said second component, wherein said tapered recess has a longitudinal axis which is laterally offset from and/or disposed at an angle to a longitudinal axis of said anchorage component thereby allowing adjustment of the second component from a first disposition of the second component relative to a predetermined reference

In a further broad form the present invention comprises;
a modular prosthesis for implantation in a joint of a skeletal frame, wherein the prosthesis includes a proximal component having a part for fixation to bone and a formation for receiving a joining member, and a distal component, wherein, the joining member engages a distal member; said distal member including a recess which receives therewithin at least one insertable element, wherein said at least one element includes means to receive and or retain said joining member; wherein said at least one element enable orthogonal, rotational and axial adjustment of said joining member.

In another broad form according to a method aspect the present invention comprises:
a method of insertion of a modular prosthesis assembly in a bone site of a skeletal frame, wherein the modular prosthesis assembly comprises; an anchorage component insertable in bone and a coupling component which co operates with said anchorage component to assume a first predetermined orientation relative to said anchorage component; the assembly further comprising an adaptor insertable between said anchorage component and said coupling component to allow a secondary adjustment of said coupling component relative to said first predetermined orientation; the method comprising the steps of
a) taking an anchorage component and inserting said component in bone;
b) taking a coupling component and placing said coupling component in engagement with said anchorage component so that the coupling component assumes a first orientation;
c) checking the first orientation of the coupling component to determine if that orientation is a desired orientation relative to a predetermined anatomical reference;
d) in the event that the first orientation is incorrect relative to said anatomical reference, removing said coupling member from engagement with said anchorage member;
e) engaging said adaptor with said anchorage member and engaging said coupling member with said adaptor;
f) adjusting said adaptor and/or said coupling member so that said coupling member assumes a secondary disposition which is a preferred orientation relative to a predetermined anatomical reference.

Preferably the adaptors include a taper, such as but not limited to a Morse which engages components having a corresponding taper.

### DESCRIPTION OF DRAWINGS

The present invention will be now described according to a preferred but non limiting embodiment and with reference to the accompanying illustrations wherein

FIG. 1 shows an underside perspective view of a typical tibial component.

FIG. 2 shows a top side view of the tibial component of FIG. 1.

FIG. 3 shows a front elevation view of the tibial component of FIG. 1.

FIG. 4 shows a side elevation view of the tibial component of FIG. 1.

FIG. 5 shows a long sectional elevation of a known tibial component inserted in a tibial stem.

FIG. 6 shows a long sectional elevation of a known tibial component inserted in a tibial stem having an angular offset.

FIG. 7 shows an exploded schematic view of a series of prosthesis assemblies in accordance with the present invention.

FIG. 8 shows a perspective view of an anchorage member

FIG. 9 shows a top view of the anchorage member of FIG. 8

FIG. 10 shows a long sectional view of the anchorage member of FIG. 8 taken at line D-D of FIG. 11.

FIG. 11 shows an elevation view of the anchorage member of FIG. 8

FIG. 12 shows a perspective view of an anchorage member with offset angular recess.

FIG. 13 shows a top view of the anchorage member of FIG. 12

FIG. 14 shows a long sectional view of the anchorage member of FIG. 12 taken at line E-E of FIG. 15.

FIG. 15 shows an elevation view of the anchorage member of FIG. 12.

FIG. 16 shows a perspective view of a neutral revision anchorage member (tibial stem).

FIG. 17 shows a top view of the anchorage member of FIG. 16

FIG. 18 shows an elevation view of the anchorage member of FIG. 16

FIG. 19 shows a long sectional elevation view of the anchorage member of

FIG. 18 taken at G-G.

FIG. 20 shows a perspective view of a revision anchorage member (tibial stem) with lateral offset.

FIG. 21 shows a top view of the anchorage member of FIG. 20

FIG. 22 shows an elevation view of the anchorage member of FIG. 20

FIG. 23 shows a long sectional elevation view of the anchorage member of FIG. 20 taken at F-F.

FIG. 24 shows a top view of an adaptor according to one embodiment with a laterally offset internal tapered cavity.

FIG. 25 shows a long sectional view of the adaptor of FIG. 24 taken at line A-A.

FIG. 26 shows a top view of an adaptor according to one embodiment with a laterally offset internal tapered cavity.

FIG. 27 shows a long sectional view of the adaptor of FIG. 24 taken at line B-B.

FIG. 28 shows a top view of an adaptor according to one embodiment with a laterally offset internal tapered cavity.

FIG. 29 shows a long sectional view of the adaptor of FIG. 24 taken at line C-C.

FIG. 30 shows an elevation view of a revision assembly including a lateral adjustment according to one embodiment of the invention.

FIG. 31 shows a long section of the assembly of FIG. 30 taken at line I-I.

FIG. 32 shows an elevation view of a revision assembly with vertical adjustment according to one embodiment of the invention.

FIG. 33 shows a top view of the assembly of FIG. 32.

FIG. 34 shows a long section of the assembly of FIG. 32 taken at line H-H.

The invention will be primarily described with reference to its application in knee prostheses. It will be appreciated however, that the assembly described herein including the use of an angular and/or lateral offset for re adjustment of a component may be applied in a variety of skeletal sites including but not limited to shoulder, ankle, finger, thumb joint. Also the assembly may be employed in dental applications. In known total knee prostheses the articular surface of the distal femur and proximal tibia are usually but not exclusively replaced with respective metal and plastic condylar-type articular bearing components. The knee prostheses provide adequate rotational and translational freedom and require minimal bone resection to accommodate the components within the boundaries of the available joint space. The patella-femoral joint may also be resurfaced by a third prosthetic component, as well.

The femoral, tibial and patella prosthetic resurfacing components are affixed to respective, surgically prepared adjacent bone structure by cementing or by biological bone ingrowth. The femoral component is usually but not exclusively a metallic alloy construction such as cobalt-chrome alloy and provides medial and lateral condylar bearing surfaces of similar shape and geometry as the natural distal femur. The tibial component can be made entirely of ultra high molecular weight polyethylene or can be comprised of a metallic base and stem component distally and an interlocking plastic (UHMWPE) component, proximally. The plastic tibial plateau bearing surfaces are of concave multi-radius geometry to more or less match the articular geometry of the mating femoral condyles, depending upon the desired design mechanics of primary femoro-tibial motion, e.g. the flexion-extension, including posterior rollback and rotational and translational articular motions.

The femoral and tibial components are positioned on the respective side of the knee joint and are not mechanically connected or linked together (unlike the case of constrained or hinged type of knee prostheses).

Additionally, in resurfacing types of total knee prostheses the tibial plateau bearing surface geometry can assume a variety of configurations, depending upon the desired extent of articular contact and associated translational (medial-lateral and anterior-posterior) and rotational (axial and varus-valgus) secondary femoro-tibial motions. These various secondary motions allow the resurfaced knee to function in a natural-like biomechanical manner in conjunction with the surrounding ligamentous and muscle structures about the knee joint. The viable soft tissue structures functionally maintain the femoral and tibial bearing surfaces in contact, provide the necessary levels of constraining force to achieve knee joint stability, and decelerate the principal motion in flexion-extension and secondary motions, such as axial rotation, etc. in a controlled manner. Additionally, this functional interaction been the surrounding tissue structures and the implanted knee prosthesis minimizes abrupt motion stoppage or impact loading of properly designed prosthetic articular surfaces, and thus prevents overstressing at the component fixation interface.

The objective in knee replacements is to simulate with a dynamic implant, natural knee function as closely as possible and any improvement which allows a surgeon greater flexibility in achieving this objective is desirable. The articulation of the femoral condyles with the tibial plateau bearing surfaces is complex biomechanics allowing primary femoro-tibial flexion and extension, and secondary motions of axial and varus-valgus rotations and anterior-posterior and medial-lateral translations, all of which occur in the normal knee joint. The knee joint reaction forces during primary or secondary motion are principally supported by the tibial bearing surfaces, and to some extent by the cam/follower surfaces, and are transferred to the underlying fixation interfaces and adjacent supportive bone structures. In a normal knee, physiological femoro-tibial rollback start at the onset of knee flexion and is generally mostly completed by 40 degrees of flexion. This rollback is accompanied by a transitional motion of rolling and sliding. In the normal knee, these complex interactions are accompanied by complex active interaction of the anterior and posterior cruciate ligaments and other surrounding adjacent soft tissue structures.

The above is a description of known biomechanics of a knee joint prosthesis.

The present invention described herein with reference to alternative embodiments, provides a prosthesis assembly including adaptors which enable a surgeon to make fine adjustments to the disposition or attitude of a component to enable that component to be disposed such that it will allow more accurate simulation of anatomical geometry or dynamic action at an implant site in a patient.

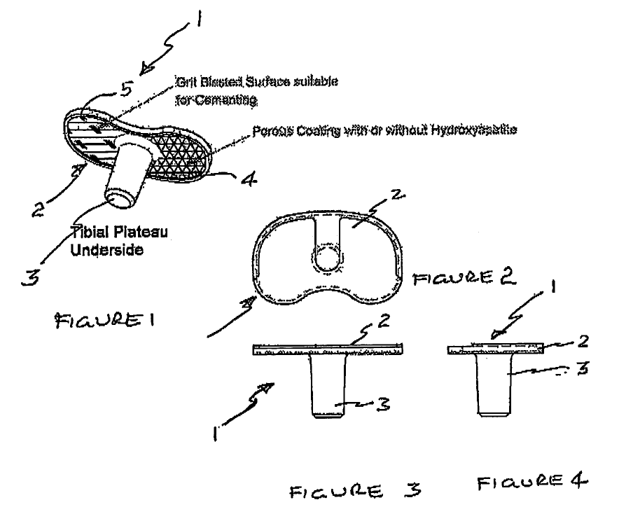

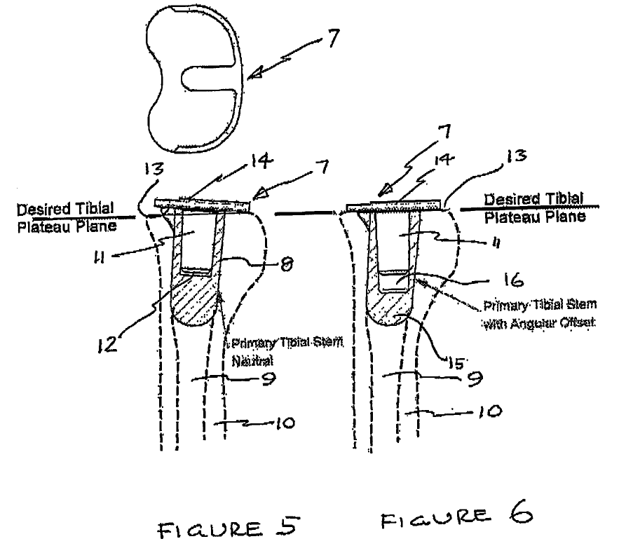

FIG. 1 shows an underside perspective view of a typical tibial component 1. Tibial component 1 comprises a tibial plate 2 and a tibial stem 3. An underside surface of plate 2 may be adapted with a porous coating 4 with or without the use of a bone growth promoter Hydroxyapatite. Alternatively, as shown with reference to surface 5 the underside surface of plate 2 may be roughened by grit blasting. FIG. 2 shows a top side view of the tibial component 1 of FIG. 1. Plate 2 includes a formation which receives and retains a polyethylene layer which provides a bearing surface for an opposing femoral component. FIG. 3 shows a front elevation view of the tibial component 1 of FIG. 1. FIG. 4 shows a side elevation view of the tibial component of FIG. 1.

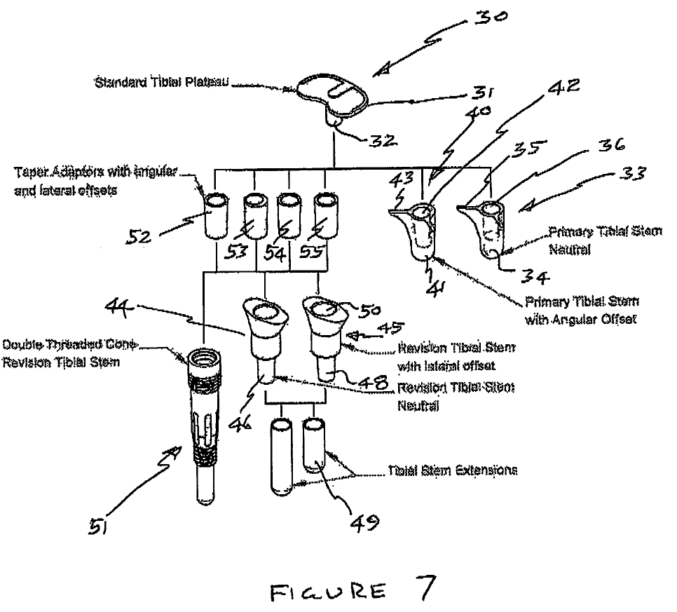

FIG. 5 shows a long sectional elevation of a known tibial component 7 inserted co axially in a tibial anchorage member 8 inserted in medullary cavity 9 of tibia 10. Stem 11 of plate 14 engages recess 12 such that a longitudinal axis of stem 11 is co axial with a longitudinal axis of recess 12. Tibia includes a resected plateau 13 which provides a reference for tibial plate 14 upon insertion in medullary cavity 9. As shown in FIG. 5 tibial plate 14 may be out of alignment with an anatomical reference such as plateau 13. In that case, where the surgeon anticipates the possibility of an out of alignment of plate 14, an anchorage with an angular offset may be used to adjust the attitude of plate 14. Referring to FIG. 6 there is shown a long sectional elevation of tibial component 7 inserted in a tibial stem having an angular offset. Tibial component 7 is inserted co axially in a tibial anchorage member 15 inserted in medullary cavity 9 of tibia 10. Stem 11 of plate 14 engages recess 16 such that a longitudinal axis of stem 11 is offset relative to a longitudinal axis of recess 16. Tibia 10 includes a resected plateau 13 which provides a reference for tibial plate 14 upon insertion in medullary cavity 9. As shown in FIG. 6 tibial plate 14 is now in alignment with plateau 13 so the optimal position of tibial plate 14 which is co planar with its reference plateau 13 will facilitate accurate simulation of joint geometry.

Thus, according to one embodiment of the invention, a surgeon is able to effect an adjustment to a component by means of an offset in provided with or in an anchorage set in a bone site.

Referring to FIG. 7 there is shown a schematic exploded layout of various components capable of use in the prosthesis assembly according to various embodiments. In the example of FIG. 7 there is shown a tibial component 30 comprising a tibial plate 31 and stem 32. Tibial stem 32 is adapted for insertion in an anchorage member 33. Anchorage member 33 comprises a body 34 including locating wings 35. Locating wings 35 allow anchorage member 33 to lock into a bone to prevent unwanted movement. Body 34 also includes a tapered recess 36 which is either co axial with or off set relative to a longitudinal axis of body 34. Anchorage member 40 is similar to anchorage member 33 except that whereas in the latter, recess 36 is co anal with a longitudinal axis of body 34, in the former, a longitudinal axis of body 41 is offset relative to a longitudinal axis of recess 42. When tapered recess 36 receives and retains therein tibial stem 32, this will dictate the orientation in situ of tibial plate 31 relative to anchorage member 33 and/or to a predetermined anatomical reference. Ideally when in situ, tibial plate 31 will be parallel with a bone plateau prepared by the surgeon prior to fixation of anchorage member 33. However, as shown in FIG. 5 this is not always the case and at present the surgeon has no expedient means to make adjustments to the orientation of the tibial plate once it has been inserted (see FIG. 5). Accurate insertion of the anchorage member 33 may be inhibited by a patients bone condition or The manner of reaming of the medullary cavity prior to insertion. Errors in reaming may be translated into an error in the disposition of tibial plate 31. In many cases the orientation of tibial plate 31 will be outside an optimum disposition for ultimate simulation by the artificial joint of natural joint geometry and function. Rather than re set anchorage member 33. The present invention allows a surgeon to make fine adjustments to improve the orientation of the tibial plate so it is set in a disposition required relative to a predetermined anatomical or other reference. According to one embodiment, the surgeon may choose an off set anchorage member 40 to receive stem 32. Offset recess 42 which also includes locating wings 43 will allow the surgeon to orient the tibial plate 31 to align with a predetermined bone plateau so ultimately the completed joint will simulate patient anatomical movement. According to an alterative embodiment, the surgeon may choose one ore more adaptors which are inserted between tibial component 30 and either primary tibial stems 33 or 40. In FIG. 7 there is shown a series of adaptors 52, 53, 54 and 55 which are available for insertion between stem 32 and either anchorage member 33 or 40. Although only four adaptors are shown it will be appreciated that a typical inventory of adaptors may be in the order of 8 or more. An adaptor may be selected to allow a surgeon to adjust the orientation of tibial plate 31 in the event that when inserted in one or other of the permanently fixed anchorages 33 or 40 the orientation of plate 31 is undesirable. Using a preselected adaptor, the surgeon may adjust the orientation and/or attitude of tibial plate 31 rotationally about X, and/or Y and/or Z axes or axially along a Z axis. The adaptors also allow lateral displacement relative to X or Y axes. An adaptor may be used to adjust the length of an implant, the gradient of tibial plate 31 the rotation about an axis through stem 32 and to off set tibial component 30 as required. Should tibial plate 31 be initially implanted with an unwanted gradient or orientation, the surgeon now has the option of adjusting the state of repose of tibial plate 31 so that it will interact with condyles of a femoral implant to more accurately simulate joint dynamics. The adaptors allow a surgeon to compensate for orientation errors in the tibial plate 31 and to eliminate the potential for uneven wear in the implanted prosthesis.

In another embodiment, alternative anchorage members are used to extend the depth of penetration inside a medullary cavity. In the case of a revision where bone has degraded an allograft may be required. This will normally necessitate a deeper anchorage in the medullary cavity. For this purpose revision tibial stems 44 or 45 may be used. Revision stem 44 includes a male tapered end 46 capable of engagement with stem extension 47. Engagement between tapered end 46 and stem extension 47 is preferably via a Morse taper and extends the prosthesis deep into a bone medullary cavity to secure adequate fixation taking into account the condition of the bone. Likewise, revision stem 45 includes a male tapered end 48 capable or engagement with stem extension 49. Engagement between tapered end 48 and stem extension 49 is preferably via a Morse taper and extends the prosthesis deep into a bone medullary cavity to secure adequate fixation taking into account the condition of the bone. Tibial stem 45 includes a lateral offset which places recess 50 out of alignment with stem 48. The offset may be required where a directional adjustment is required proximally.

In an alternative embodiment, in order to achieve anchorage extension a double threaded cone 51 may be employed. Cone 51 includes recess 52 which receives therein one of adaptors 52, 53, 54 and 55.

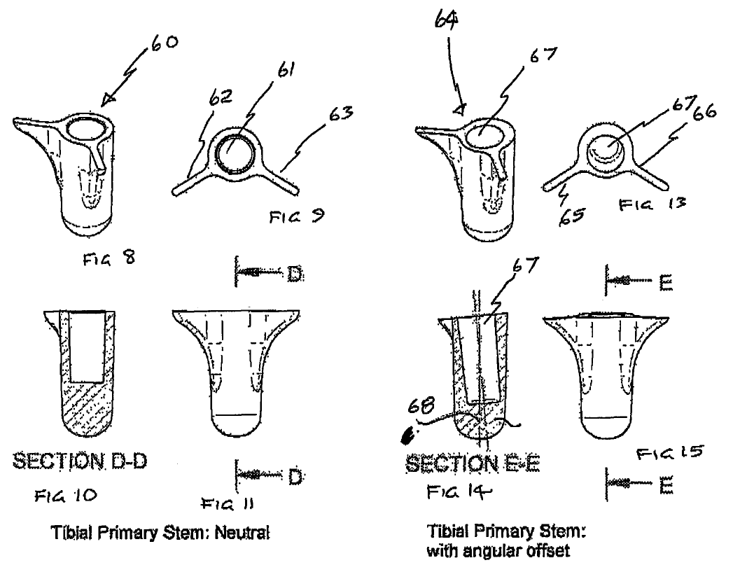

FIG. 8 shows a perspective view of an anchorage member 60 capable of insertion in a medullary cavity of a bone. FIG. 9 shows a top view of anchorage member 60. Member 60 includes a tapered recess 61 and locating wings 62 and 63 which resist unwanted rotation in a cavity in which member 60 is inserted.

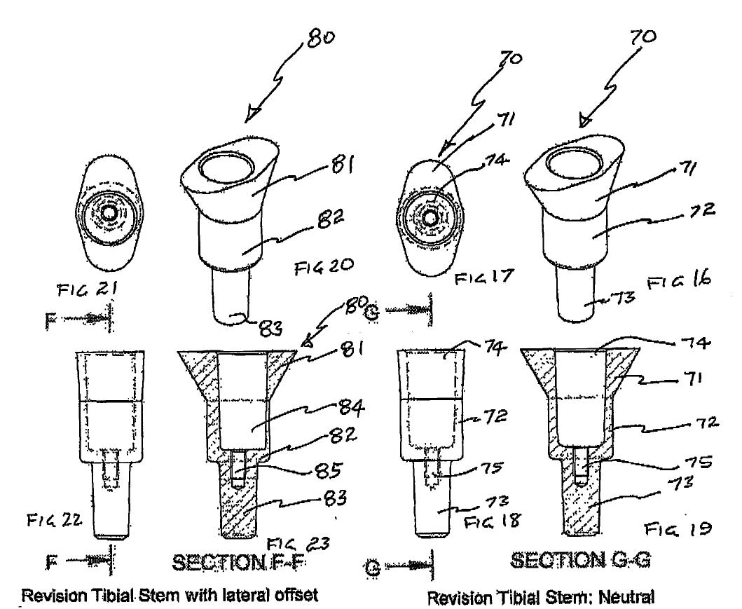

FIG. 10 shows a long sectional view of the anchorage member 60 taken at line D-D of FIG. 11

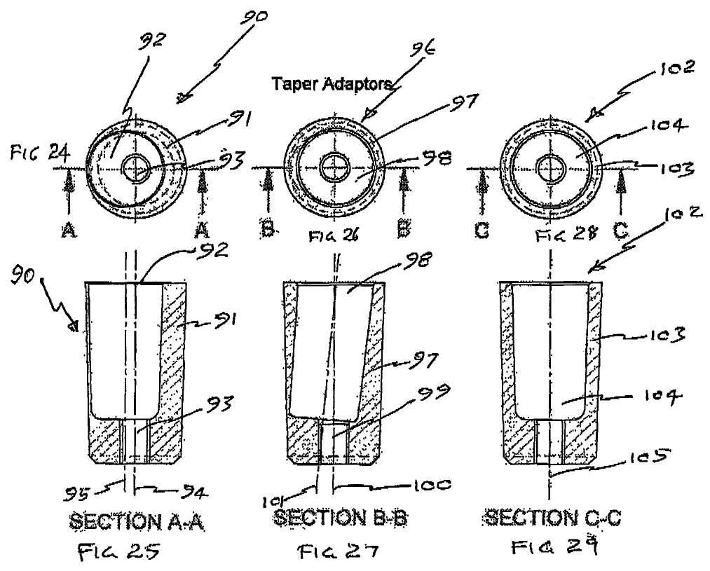

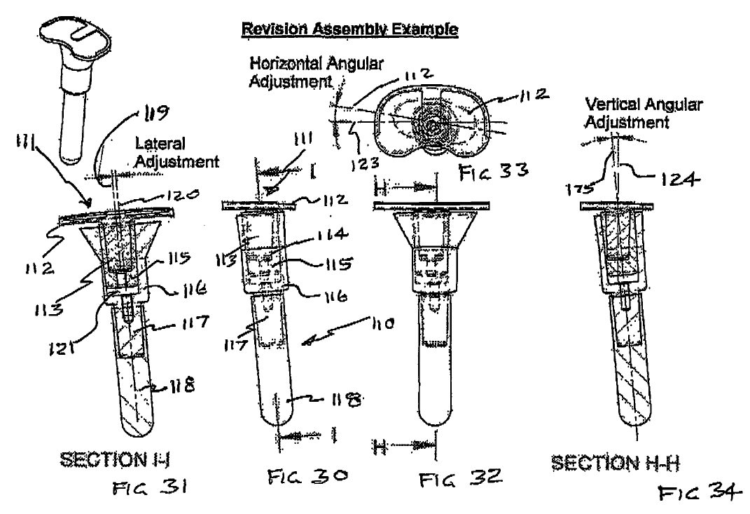

FIG. 12 shows a perspective view of an anchorage member 64 including locating wings 65 and 66 and offset angular recess 67. FIG. 13 shows a top view of the anchorage member of FIG. 12 and FIG. 14 shows a long sectional view of anchorage member 64 taken at line E-E of FIG. 15. Offset recess 67 as shown in FIG. 14 is disposed at a predetermined angle relative to longitudinal axis 68. Recess 67 receives and retains therein a tibial component such as that shown and described in FIGS. 1-4. Offset recess 67 allows the surgeon to set a tibial plate closer to a predetermined reference. elect use of an offset adjust the attitude of a tibial plate. FIG. 16 shows a perspective view of a neutral revision anchorage member 70 (tibial stem) according to one embodiment. Member 70 is preferred for revision operations requiring allograft bone and comprises an elongated body comprising a flared collar 71, waist 72 and tapered stem 73. FIG. 17 shows a top view of the anchorage member of FIG. 16. Flared collar 71 includes a recess 74 which receives and retains therein stem 73. FIG. 1I shows an elevation view of the anchorage member of FIG. 16 and FIG. 19 shows a long sectional elevation view of the anchorage member 70 of FIG. 18 taken at G-G. Stem 73 locates in recess 74. An adaptor (see FIGS. 24-29) may be secured within recess 74 by means of a screw which penetrates recess 75. Anchorage member 70 is characterised in that a longitudinal axis of recess 74 is co axial with a longitudinal axis of stem 73. FIG. 20 shows a perspective view of a revision anchorage member 80 (tibial stem) with lateral offset. Member 80 is preferred for insertion in a medullary cavity in revision operations requiring allograft bone where deeper penetration is required. Anchorage member and comprises an elongated body comprising a flared collar 81, waist 82 and tapered stem 83. FIG. 21 shows a top view of the anchorage member of FIG. 20. Flared collar 81 includes a recess 84 which receives and retains therein stem 83. FIG. 22 shows an elevation view of the anchorage member of FIG. 20 and FIG. 23 shows a long sectional elevation view of the anchorage member 80 of FIG. 22 taken at line F-F. Stem 33 locates in recess 84. An adaptor (see FIGS. 24-29) may be secured within recess 84 by means of a screw which penetrates recess 85. Anchorage member 80 is characterised in that a longitudinal axis of recess 84 is laterally offset relative to a longitudinal axis of stem 83. Respective recesses 74 and 84 of (tibial stem) anchorage members 70 and 80 may receive an adaptor of the type described in FIGS. 24-29. These adaptors may also be used in conjunction with anchorage members 60 and 64 previously described.

FIG. 24 shows a top view of an adaptor 90 according to one embodiment comprising a body 91 wilt a laterally offset internal tapered cavity 92. FIG. 25 shows a long sectional view of adaptor 90 of FIG. 24 taken at line A-A. Adaptor 90 includes a passage 93 which allows insertion of a screw for fixation of adaptor 90 to an anchorage such as those described in FIGS. 8, 12, 16, 20. Longitudinal axis 94 is laterally displaced from but parallel to longitudinal axis 95 such that when adaptor 90 is inserted in an anchorage member, a coupling member (not shown) inserted in recess 92 will be laterally displaced from an otherwise neutral position. A fine lateral adjustment may be an advantage for an implant which is not initially disposed in an optimal alignment.

FIG. 26 shows a top view of an adaptor 96 according to one embodiment with a laterally offset internal tapered cavity. FIG. 27 shows a long sectional view of the adaptor of FIG. 26 taken at line B-B. Adaptor 96 includes a passage 99 which allows insertion of a screw for fixation of adaptor 96 to an anchorage such as those described in FIGS. 8, 12, 16, 20. Longitudinal axis 100 is at an angle to longitudinal axis 101 such that when adaptor 96 is inserted in an anchorage member, a coupling member (not shown) inserted in recess 98 will be disposed at an angle from an otherwise neutral position. A fine lateral adjustment may be an advantage for an implant which is not initially disposed in an optimal alignment. FIG. 28 shows a top view of an adaptor 102 according to one embodiment with a body 103 having internal tapered cavity 104. Cavity 104 is in axial alignment with a longitudinal axis 105 of adaptor 102, FIG. 29 shows a long sectional view of the adaptor 102 of FIG. 24 taken at line C-C.

FIGS. 30-34 show examples of adjustments which may be made using a revision prosthesis assembly. Shown by way of example are lateral, horizontal angular and vertical angular adjustments which a surgeon may make in a revision assembly. FIG. 30 shows an elevation view of a revision assembly 110 according to one embodiment of the invention. Assembly 110 comprises a tibial component 111 comprising a tibial plate 112 and a tibial stem 113. Tibial stem 113 locates in internal tapered recess 114 of adaptor 115. Adaptor 115 accommodates stem 113 via means of interfitting tapers. The assembly 110, includes tibial revision stem 116 which engages via tapered end 117 an extension member 118. FIG. 31 shows a long section of the assembly of FIG. 30 taken at line I-L. As may be seen from FIG. 31 a longitudinal axis 119 of adaptor 115 is laterally offset from longitudinal axis 120 of extension tibial vision stem (anchorage) 116 and extension 118. As shown in FIG. 31, laterally offset recess 121 may be combined with another offset cavity in adaptor 115 so that within that assembly there is a range of adjustment. Thus there may typically be anywhere between 1-6 mm of lateral adjustment depending upon how adaptor 15 is located in offset tapered recess 121. This range may vary (decrease or increase) according to the size of the components.

FIG. 32 shows an elevation view of a revision assembly similar to that shown in FIG. 31, according to one embodiment of the invention. FIG. 33 shows a top view of the assembly of FIG. 32 indicating relative to axes 122 and 123 available horizontal angular adjustment of tibial plate 112. FIG. 34 shows a long section of the assembly of FIG. 32 taken at line H-H. Reference axes 124 and 125 indicate available angular adjustment enabled by angular offset adaptor 115. The revision assembly may be adjusted by selection of adaptors such as those shown in FIGS. 24-29. Tapers enabling fitting of adaptors to an anchorage member are preferably Morse tapers. The arrangements described above with reference to a tibial component are adaptable also to a corresponding femoral component of a knee prosthesis.

Typically a femoral component includes a proximal shaft member for insertion in a medullary cavity of a femur. According to one embodiment, the shaft may be a known double threaded cone (Margron ™) for compressive fixation. The proximal shaft includes a tapered recess which receives a joining element. The femoral component further comprises a distal element having a recess which receives and retains an adaptor hereinbefore described. This effectively provides a taper within a taper and allows the ability to fit a fixture on the taper thereby allowing adjustment by rotation, offset, vertical height and horizontal adjustment in three dimensions (i.e. relative to XY&Z axes.

For any joint prosthesis replacement including the knee to function optimally 4 vectors need to be considered in the design to return the joint position in space to as normal as possible a natural position. The four vectors are;
1 medial-lateral
2 anterior-posterior
3 rotational
4 vertical height

The four axis double taper arrangement allows for correction in all 4 degrees of freedom to accomplish that objective. The jointing arrangement described above using an offset taper within a taper will assist a surgeon in finding appropriate joint references accurately such as the horizontal line.

The inserts described herein may be manufactured from Chrome cobalt or Titanium.

It will be recognised by persons skilled in the art that numerous variations and modifications may be made to the invention broadly described herein without departing from the overall spirit and scope of the invention.

1. A prosthesis assembly for implantation in a skeletal site: the assembly comprising; a first component for fixation, in. a bone cavity, a second component capable of direct or indirect engagement with the first component; at least one adaptor which engages said first and second components thereby allowing adjustment of the second component from a first disposition of the second component relative to a predetermined reference.

2. A prosthesis assembly according to claim 1 wherein the first component provides an anchorage in said bone for the assembly and receives said at least one adaptor.

3. A prosthesis assembly according to claim 2 wherein the at least one adaptor joins the first component to the second component.

4. A prosthesis assembly according to claim 3 wherein said joining adaptor includes a body having an external tapered region and a tapered inner recess.

5. A prosthesis assembly according to claim 4 wherein said external tapered region releasably engages said first component

6. A prosthesis assembly according to claim 5 wherein, said inner tapered recess receives therein said second component.

7. A prosthesis assembly according to claim 6 wherein, the second component is adjustable through at least four degrees of freedom relative to said reference.

8. A prosthesis according to claim 7 wherein each said at least one adaptor allows adjustment of second component laterally, angularly, axially or rotationally relative to x, y and z axes.

9. A prosthesis assembly according to claim 8 wherein the external tapered region is symmetric relative to a longitudinal axis of said adaptor

10. A prosthesis assembly according to claim 9 wherein the inner taper of the adaptor is co axial with the external taper.

11. A prosthesis assembly according to claim 9 wherein a longitudinal axis of the inner taper is disposed at an angle to a longitudinal axis of said adaptor.

12. A prosthesis assembly according to claim 9 wherein, said inner taper is offset relative to but parallel to a longitudinal axis of said adaptor.

13. A prosthesis assembly according to claim 9 wherein said inner taper is offset from and at an angle relative to a longitudinal axis of the adaptor.

14. A prosthesis assembly according to claim 1 wherein said first component, includes a tapered recess which engages said external taper of said adaptor.

15. A prosthesis assembly according to claim 14 wherein said second component comprises a tibial plate connected to a tapered stem.

16. A prosthesis assembly according to claim 14 wherein the first component is a femoral implant.

17. A prosthesis according to claim 15 wherein said adaptor allows adjustment of an angle of repose of said tibial plate.

18. A modular prosthesis assembly comprising; an anchorage component insertable in bone and a coupling component which cooperates with said anchorage component to assume a first predetermined orientation relative to said anchorage component; the assembly further comprising an adaptor insertable between said anchorage component and said coupling component to allow a secondary adjustment of said coupling component relative to said first predetermined orientation of said coupling component.

19. An assembly according to claim 18 wherein the anchorage member and the coupling member are capable of engagement with each other via male/female or female/male tapers.

20. An assembly according to claim 19 wherein said adaptor is engageable with said anchorage member and said coupling member via male/female or female/male tapers.

21. An adaptor for use with a prosthesis assembly for implantation in a skeletal site, the adaptor including a body having an external tapered region and an inner tapered recess, and wherein said external, tapered region engages a corresponding tapered recess of a first implantable component of said assembly and the inner tapered recess receives therein a second component of said assembly.

22. An adaptor according to claim 11 wherein the adaptor allows adjustment of said second component relative to a first engaged position of said second component.

23. An adaptor according to claim 22 wherein the adaptor is cylindrical.

24. An adaptor according to claim 23 wherein the inner taper is co axial with the outer taper.

25. An adaptor according to claim 24 wherein a longitudinal axis of the inner taper is disposed at an angle to a longitudinal axis of said adaptor.

26. An adaptor according to claim 25 wherein said inner taper is offset from but parallel to a longitudinal axis of said adaptor.

27. An adaptor according to claim 26 wherein said taper is offset from and at an angle relative to a longitudinal axis of the adaptor.

28. An adaptor according to claim 27 wherein said first component provides and anchorage for said second component

29. A knee prosthesis comprising a femoral component for attachment to a femur, an opening defined by the femoral component, a tibial component for attachment to a tibia, an opening through the tibial component, a bearing component between the femoral and tibial components, the femoral component and the bearing component having respective curved articulatory bearing surfaces; the knee prosthesis further comprising an adaptor capable of use with said tibial of femoral component; wherein said adaptor enables secondary orthogonal, rotational lateral and axial adjustment of said tibial and femoral components.

30. A prosthesis assembly for implantation in a skeletal site; the assembly comprising; a first anchorage component for fixation in a bone cavity, a second component capable of direct or indirect engagement the first component; wherein, the anchorage component comprises a tapered recess which receives a corresponding tapered member of said second component; wherein said tapered recess has a longitudinal axis which is laterally offset from and/or disposed at an angle to a longitudinal axis of said anchorage component thereby allowing adjustment of the second component from a first disposition of the second component relative to a predetermined reference.

31. A method of insertion of a modular prosthesis assembly in a bone site of a skeletal frame, wherein the modular prosthesis assembly comprises; an anchorage component insertable in bone and a coupling component which co operates with said anchorage component to assume a first predetermined orientation relative to said anchorage component; the assembly further comprising an adaptor insertable between said anchorage component and said coupling component to allow a secondary adjustment of said coupling component relative to said first predetermined orientation; the method comprising the steps of a) taking an anchorage component and inserting said component in bone; b) taking a coupling component and placing said coupling component in engagement with said anchorage component so that the coupling component assumes a first orientation; c) checking the first orientation of the coupling component to determine if that orientation is a desired orientation relative to a predetermined anatomical reference; d) in the event that the first orientation is incorrect relative to said anatomical reference, removing said coupling member from engagement with said anchorage member; e) engaging said adaptor with said anchorage member and engaging said coupling member with said adaptor; adjusting said adaptor and/or said coupling member so that said coupling member assumes a secondary disposition which is a preferred orientation relative to a predetermined anatomical reference.
